(19) United States
(12) Patent Application Publication
(10) Pub. No.: US 2008/0103500 A1
(43) Pub. Date: May 1, 2008

(54) Instruments and Methods For Smoothing A Portion of A Spinal Rod

(76) Inventors: Nam Chao, Marlborough, MA (US); Anne Drzyzga, Taunton, MA (US); Nicholas Pavento, Dorchester, MA (US); Peter Newton, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/670,025

(22) Filed: Feb. 1, 2007

Related U.S. Application Data
(60) Provisional application No. 60/863,690, filed on Oct. 31, 2006.

(51) Int. Cl. A61B 17/56

(52) U.S. Cl. 606/61

(57) ABSTRACT

An instrument for smoothing an end of a spinal rod includes a housing having an opening sized and shaped to receive a spinal rod and a smoothing surface positioned within the housing and in communication with the opening. The smoothing surface has one or more structures configured to engage a portion of the spinal rod and, upon relative rotation of the spinal rod to the smoothing surface, to smooth the portion of the spinal rod by removing material from the spinal rod.

### CROSS-REFERENCE TO RELATED APPLICATIONS

This application claims priority to U.S. Provisional Patent Application No. 60/863,690 filed Oct. 31, 2006, which is incorporated herein by reference.

### BACKGROUND

During spinal surgery to treat spinal disease, for example, to correct a spinal deformity or to treat degenerative disc disease, one or more spinal rods may be cut by the surgeon or operating room staff to a length suitable for the patient's anatomy and for the procedure being performed. Frequently, the cut end of the spinal rod may have sharp edges and/or burrs that pose a safety risk to the surgeon and the operating room staff. For example, the sharp edge or burrs of the rod may cut the surgical glove or skin of the surgeon thereby exposing the surgeon and the patient to blood borne pathogens. Accordingly, there is a need for an instrument that can smooth the edges of the end of a spinal rod.

### SUMMARY

Disclosed herein are instruments and methods for smoothing a portion of a spinal rod. In one exemplary embodiment, an instrument for smoothing an end of a spinal rod may include a housing having an opening sized and shaped to receive a spinal rod and a smoothing surface positioned within the housing and in communication with the opening. The smoothing surface may have one or more structures configured to engage a portion of the spinal rod and, upon relative rotation of the spinal rod to the smoothing surface, to smooth the portion of the spinal rod by removing material from the spinal rod.

In another exemplary embodiment, an instrument for smoothing an end of a spinal rod may include a cylindrical housing having a rod opening at a first end thereof and an approximately conical smoothing surface positioned within the housing and in communication with the rod opening. The smoothing surface may have one or more cutting blades configured to engage an end of the spinal rod and, upon relative rotation of the spinal rod to the smoothing surface, to smooth the end of the spinal rod by removing material from the spinal rod.

In a further exemplary embodiment, an instrument for smoothing an end of a spinal rod may include a cylindrical housing having a first rod opening at a first end thereof, a first shaft positioned in the housing, the first shaft having a first smoothing surface positioned at a first end of the first shaft, and a second shaft positioned in the housing, the second shaft having a second smoothing surface positioned at a first end of the second shaft. The first smoothing surface and the second smoothing surface may be positioned within the housing to receive an end of a spinal rod therebetween and in communication with the first rod opening. The first and second smoothing surfaces may have one or more cutting blades configured to engage the end of the spinal rod and, upon relative rotation of the spinal rod to the first and second smoothing surfaces, to smooth the end of the spinal rod by removing material from the spinal rod.

### BRIEF DESCRIPTION OF THE FIGURES

These and other features and advantages of the instrument and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1 is an exploded perspective view of an exemplary embodiment of an instrument for smoothing an end of a spinal rod;

FIG. 2 is a perspective view of the instrument of FIG. 1, illustrating the cover engaged with the housing of the instrument;

FIG. 3 is a side view of the instrument of FIG. 1;

FIG. 4 is a side view in cross section of the instrument of FIG. 1;

FIG. 5 is a perspective view of an exemplary embodiment of an instrument for smoothing an end of a spinal rod;

FIG. 6 is a side view of the instrument of FIG. 5;

FIG. 7 is an end view of the first end of the instrument of FIG. 5;

FIG. 8 is an end view of the second end of the instrument of FIG. 5;

FIG. 9 is a perspective view of a first shaft of the instrument of FIG. 5;

FIG. 10 is a side view of a first shaft of the instrument of FIG. 5;

FIG. 11 is an end view of the first shaft of the instrument of FIG. 5; and

FIG. 12 is a perspective view of the instrument of FIG. 5, illustrating the instrument with a portion of the housing removed.

### DETAIL DESCRIPTION OF EXEMPLARY EMBODIMENTS

Certain exemplary embodiments will now be described to provide an overall understanding of the principles of the structure, function, manufacture, and use of the instruments and methods disclosed herein. One or more examples of these embodiments are illustrated in the accompanying drawings. Those of ordinary skill in the art will understand that the instruments and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

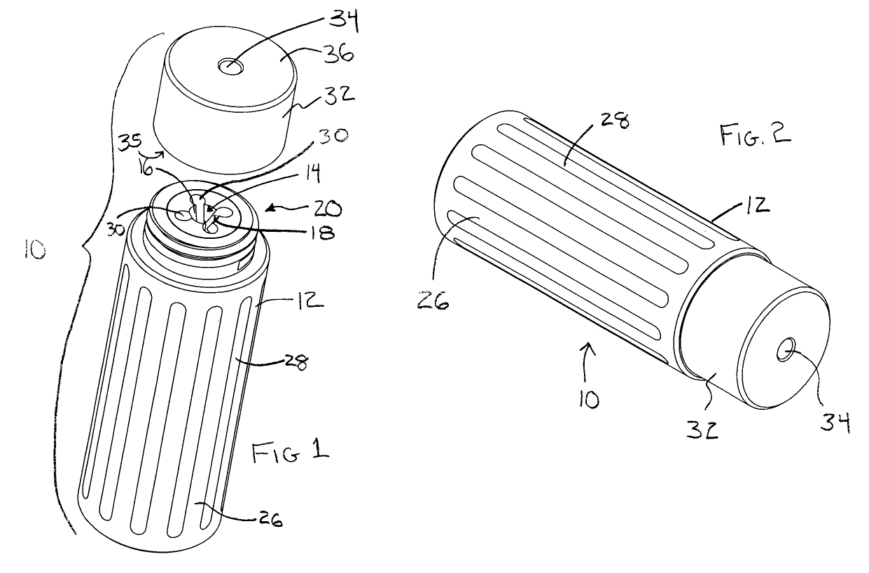

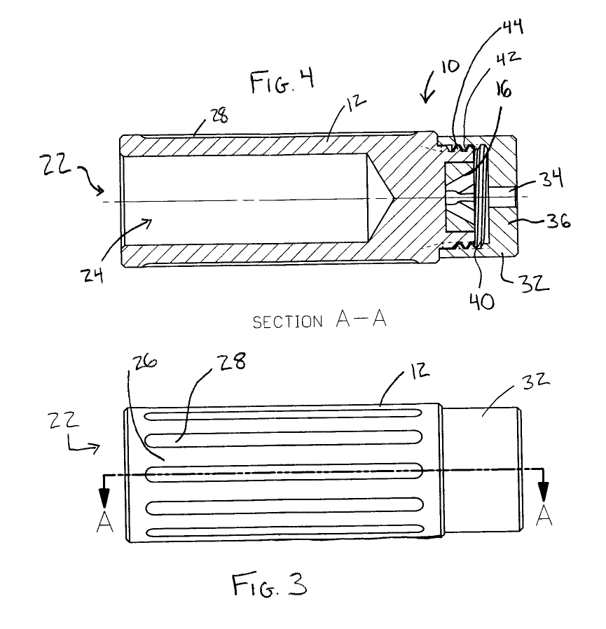

FIGS. 1-4 illustrate an exemplary embodiment of an instrument 10 for smoothing a portion, for example, an end, of a spinal rod. The exemplary instrument 10 may include a housing 12 having a rod opening 14 sized and shaped to receive a spinal rod. A smoothing surface 16 may be positioned within the housing 12. The smoothing surface 16 may be in communication with the rod opening 14 to facilitate the positioning of a portion of a spinal rod through the opening and into contact with the smoothing surface 16. The smoothing surface 16 may have one or more structures 18 configured to engage a portion of the spinal rod and, upon relative rotation of the spinal rod to the smoothing surface, to smooth the portion of the spinal rod by removing material from the spinal rod.

In the exemplary embodiment, the housing 12 of the instrument 10 is generally cylindrical in shape having a generally circular cross section, although, one skilled in the art will appreciate that other shapes, including rectilinear or oval, are possible. The housing has a first end 20 and a second end 22. The rod opening 14 is positioned in the first end 20 of the housing 12. An elongated bore 24 may be provided in the second end 22 and may extend along a portion of the housing to reduce the weight of the housing 12. The outer surface 26 may include texturing, grooves, or other surface features 28 to facilitate gripping of the housing during rod smoothing operations.

The smoothing surface 16 may have a size and shape to receive and engage a portion, for example, the end, of a spinal rod. In the exemplary embodiment, the smoothing surface 16 is approximately conical in shape and defines a rod receiving cavity having an approximately conical shape for receiving the spinal rod. In this manner, the smoothing surface 16 tapers from the rod opening 14 to the interior of the rod receiving cavity thereby allowing the smoothing surface to engage rods of varying diameters.

The smoothing surface 16 may include one or more structures 18 configured to engage and smooth a portion of a spinal rod. The structures 18 may be one or more cutting blades formed in the smoothing surface 16. The cutting blades may be edged, sharpened and/or peaked projections provided on the smoothing surface 16. In the exemplary embodiment, the structures 18 are formed be a plurality of arcuate, in cross section, slots 30 provided in the smoothing surface 16 whereby the edges of the slots 30 may engage and smooth a portion of the rod during rotation of the rod relative to the smoothing surface 16. The slots 30 may be spaced about the smoothing surface 16 and any number of slots 30, or other structures, may be provided. The smoothing surface 16 and the structures 18 may be manufactured from any material suitable for cutting, grinding, or otherwise smoothing a metal (e.g., stainless steel or titanium) spinal rod. Suitable materials include, but are not limited to, stainless steel and stone (e.g., a grinding stone).

In the exemplary embodiment, the instrument 10 may include a removable and replaceable cover 32 engagable with the first end 20 of the housing 12. The cover 32 includes a cover opening 34 aligned with the rod opening 14 upon engagement of the cover 32 to the housing 12. The cover 32 may have a size and shape complementary to the size and shape of the housing. In the exemplary embodiment, the cover 32 is generally cylindrical in shape and has an open first end 35 and a closed second end 36 with the cover opening 34 formed in the closed second end 36. The closed second end 36 is spaced apart from the rod opening 14 upon engagement of the cover 32 with the housing 12 providing a collecting space 40 for collecting shavings, debris, or other removed material during smoothing operations. Moreover, by spacing the second end 36, and, thus, the cover opening 34, from the rod opening 14, the cover 32 may operate to stabilize the rod during smoothing operations. The cover opening 34 may be sized and shaped to approximate the size and shape of the spinal rod to facilitate stabilization of the spinal rod during smoothing operations. For example, in the case of round spinal rods, the cover opening 34 may have a diameter approximate to standard sizes for round spinal rods, such as, for example, a rod diameter of approximately 3.0 mm, 3.5 mm, 4.0 mm, 4.5 mm, 5.0 mm, 5.5 mm, or 6.35 mm. In the case of a 5.5 mm spinal rod, for example, the cover opening 32 may be 6.0 mm in diameter and in the case of a 6.35 mm spinal rod, for example, the cover opening 32 may be 6.85 mm in diameter. One skilled in the art will appreciate that the cover opening 34 is not limited to these sizes or a round shape.

The cover 32 may include a connection mechanism to permit selective connection of the cover 32 to the housing 12 of the instrument 10. In the illustrated embodiment, for example, the cover 32 includes internal threads 42 for engagement with external threads 44 of the first end of housing 12. In other embodiments, alternative connection mechanisms, for example a snap-fit connection, may be employed.

In certain exemplary embodiments, the instrument 10 may include additional removable and replaceable covers having cover openings of differing sizes and/or shapes. Such additional covers facilitate the use of one instrument 10 with rods of differing sizes and shapes.

The instrument 10 may be manufactured from metal, polymers, ceramics, or other material suitable for use in medical instruments. In one exemplary embodiment, the instrument 10 is manufactured from stainless steel.

In use, a portion of a spinal rod, e.g., an end of a spinal rod, may be inserted through the cover opening 34 and the rod opening 14 into engagement with the smoothing surface 16. By rotating the spinal rod relative to the smoothing surface 16, the rod is smoothed. In certain embodiments, the rod 12 may be rotated relative to the housing 12. In other embodiments, the housing 12 may be rotated relative to the rod. The housing 12 may be configured for connection to a rotary drive, e.g., a rotating shaft, of a power tool to facilitate rotation of the housing 12 relative to the spinal rod.

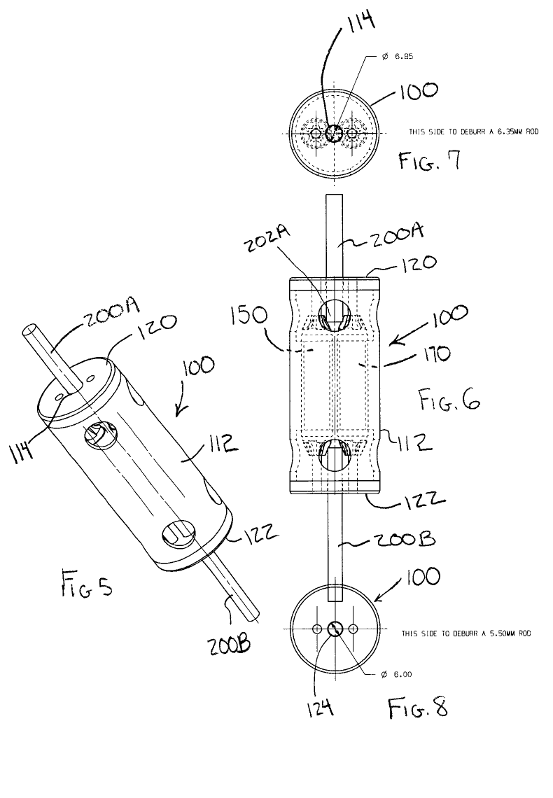

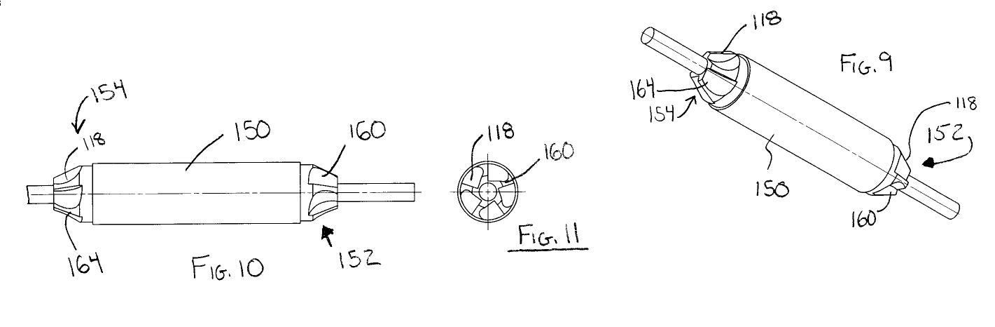

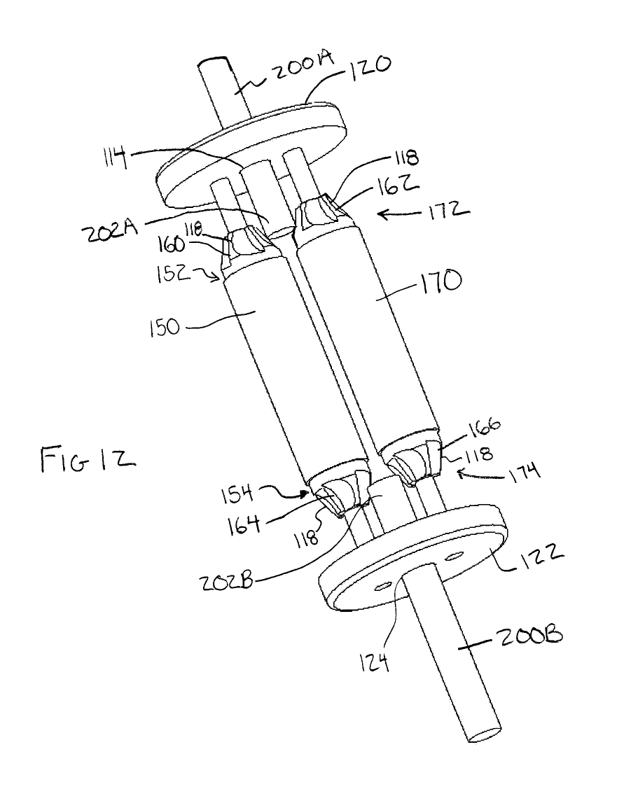

FIGS. 5-12 illustrate an other embodiment of an instrument 100 for smoothing an end of one or more spinal rods 200A,B. The instrument 100 may include a cylindrical housing 112 having a first rod opening 114 at a first end 120 thereof, a first shaft 150 positioned in the housing 112, the first shaft 150 having a first smoothing surface 160 positioned at a first end 152 of the first shaft 150, and a second shaft 170 positioned in the housing 112, the second shaft 170 having a second smoothing surface 162 positioned at a first end 172 of the second shaft 170. The first smoothing surface 160 and the second smoothing surface 162 may be positioned within the housing 112 to receive an end 202A of a spinal rod 200A therebetween and in communication with the first rod opening 114. The first and second smoothing surfaces 160,162 may have one or more cutting blades 118 configured to engage the end 202A of the spinal rod 200A and, upon relative rotation of the spinal rod 200A to the first and second smoothing surfaces 160, 162, to smooth the end 202A of the spinal rod 200A by removing material from the spinal rod 200A.

The housing 112, in the exemplary embodiment, includes a second end 122 having a second rod opening 124 formed therein for receiving a second spinal rod 200B. The second rod opening 124 may have a size and a shape distinct from the first rod opening 114. In the exemplary embodiment, for example, the first rod opening 114 may be sized to receive a round spinal rod 200A having a 6.35 mm diameter and the second rod opening 124 may be sized to receive a round spinal rod 200B having a 5.5 mm diameter. The first rod opening 114 and the second rod opening 124 may have a size approximate the size of the spinal rod to be received therein. For example, the first rod opening 114 has a diameter of 6.85 mm to receive a 6.35 mm spinal rod 200A and the second rod opening has a diameter of 6.0 to receive a 5.5 mm spinal rod 200B.

In the exemplary embodiment, the first shaft 150 may have a third smoothing surface 164 positioned at a second end 154 of the first shaft 150 and the second shaft 170 has a fourth smoothing surface 166 positioned at a second end 174 of the second shaft 170, the third smoothing surface 164 and the fourth smoothing surface 166 positioned within the housing 112 to receive an end of a second spinal rod 200B therebetween and in communication with the second rod opening 124. The third and fourth smoothing surfaces 164, 166 may have one or more cutting blades 118 configured to engage the end 202B of the spinal rod 200B and, upon relative rotation of the spinal rod 200B to the third and fourth smoothing surfaces 164, 166, to smooth the end 202B of the spinal rod 200B by removing material from the spinal rod 202B.

The first shaft 150 and the second shaft 170 may be fixed to the housing 112. In the exemplary embodiment, for example, the first and second shafts 150, 170 may be connected to the ends 120, 122 of the housing 112. In alternative embodiments, the first shaft 150 and/or the second shaft 170 may rotate about their respective longitudinal central axis relative to the housing 112. In certain embodiments, the first shaft 150 may be coupled to the second shaft 170 by one or more gears that permit the first and second shafts 150, 170 to rotate together. The first and second shafts 150, 170 may be connected to a rotating shaft, gear, or other device that is rotated by a motor, for example, the motor of a rotary power tool.

The smoothing surfaces 160, 162, 164, and 166 may be an approximately conical in shape or may otherwise taper from an end of the respective shaft toward the middle of the respective shaft. In this arrangement, the first smoothing surface 160 and the second smoothing surface 162 are positioned relative to one another to create an approximately conical cavity therebetween for receiving the end 202A of the spinal rod 200A. Likewise, the third smoothing surface 164 and the fourth smoothing surface 164 are positioned relative to one another to create an approximately conical cavity therebetween for receiving the end 202B of the spinal rod 200B.

While the instruments and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. An instrument for smoothing a portion of a spinal rod, the instrument comprising:
a housing having an opening sized and shaped to receive a spinal rod; and
a smoothing surface positioned within the housing and in communication with the opening, the smoothing surface having one or more structures configured to engage a portion of the spinal rod and, upon relative rotation of the spinal rod to the smoothing surface, to smooth the portion of the spinal rod by removing material from the spinal rod.

2. The instrument of claim 1, further comprising a shaft positioned in the housing, the smoothing surface positioned proximate an end of the shaft.

3. The instrument of claim 2, wherein the shaft is fixed relative to the housing.

4. The instrument of claim 2, wherein at least a portion of the shaft is rotatable about a longitudinal central axis of the shaft.

5. The instrument of claim 2, wherein the smoothing surface is approximately conical in shape.

6. The instrument of claim 1, wherein the smoothing surface is approximately conical in shape and defines a cavity having a generally conical shape for receiving the spinal rod.

7. The instrument of claim 1, wherein the structure on the smoothing surfaces is a cutting blade.

8. An instrument for smoothing an end of a spinal rod, the instrument comprising:
a cylindrical housing having a rod opening at a first end thereof, the rod opening sized and shaped to receive a spinal rod; and
an approximately conical smoothing surface positioned within the housing and in communication with the rod opening, the smoothing surface having one or more cutting blades configured to engage an end of the spinal rod and, upon relative rotation of the spinal rod to the smoothing surface, to smooth the end of the spinal rod by removing material from the spinal rod.

9. The instrument of claim 8, further comprising a first removable and replaceable cover engageable with the first end of the housing, the first cover including a first cover opening aligned with the rod opening upon engagement of the first cover with first end of the housing.

10. The instrument of claim 9, wherein the first cover is generally cylindrical in shape having an open first end and a closed second end, the first cover opening provided in the closed second end.

11. The instrument of claim 10, wherein the closed second end is spaced apart from the rod opening upon engagement of the first cover with first end of the housing.

12. The instrument of claim 9, further comprising a second removable and replaceable cover engageable with the first end of the housing, the second cover including a second cover opening aligned with the rod opening upon engagement of the cover with first end of the housing, the second cover opening having a size distinct from the first cover opening.

13. The instrument of claim 12, wherein first cover opening is sized to receive a spinal rod having a diameter of 3.0 mm, 3.5 mm, 4.0 mm, 4.5 mm, 5.0 mm, 5.5 mm, or 6.35 mm.

14. An instrument for smoothing an end of a spinal rod, the instrument comprising:
a cylindrical housing having a first rod opening at a first end thereof, the first rod opening sized and shaped to receive a spinal rod;
a first shaft positioned in the housing, the first shaft having a first smoothing surface positioned at a first end of the first shaft; and
a second shaft positioned in the housing, the second shaft having a second smoothing surface positioned at a first end of the second shaft, the first smoothing surface and the second smoothing surface positioned within the housing to receive an end of a spinal rod therebetween and in communication with the first rod opening, the first and second smoothing surfaces having one or more cutting blades configured to engage the end of the spinal rod and, upon relative rotation of the spinal rod to the first and second smoothing surfaces, to smooth the end of the spinal rod by removing material from the spinal rod.

15. The instrument of claim 14, wherein the first shaft and the second shaft are fixed relative to the housing.

16. The instrument of claim 14, wherein at least a portion of the first shaft and at least a portion of the second shaft are rotatable about a longitudinal central axis of the first shaft and the second shaft, respectively.

17. The instrument of claim 14, wherein the first smoothing surface is approximately conical in shape.

18. The instrument of claim 14, wherein the housing has a second rod opening at a second end thereof, the second rod opening sized and shaped to receive a spinal rod and having a size distinct from the first rod opening.

19. The instrument of claim 18, wherein the first shaft has a third smoothing surface positioned at a second end of the first shaft and the second shaft has a fourth smoothing surface positioned at a second end of the second shaft, the third smoothing surface and the fourth smoothing surface positioned within the housing to receive an end of a spinal rod therebetween and in communication with the second rod opening, the third and fourth smoothing surfaces having one or more cutting blades configured to engage the end of the spinal rod and, upon relative rotation of the spinal rod to the third and fourth smoothing surfaces, to smooth the end of the spinal rod by removing material from the spinal rod.
